(19) United States
(12) Patent Application Publication
(10) Pub. No.: US 2017/0292102 A1
(43) Pub. Date: Oct. 12, 2017

(54) PACKAGING AND TRANSFER SYSTEM FOR MICROCARRIERS

(71) Applicant: CORNING INCORPORATED, Corning, NY (US)

(72) Inventors: Elizabeth Jacob Abraham, Andover, MA (US); Terry Thomas Goodrich, Arundel, ME (US); Paul Kevin Seeto, Clinton, MA (US)

(21) Appl. No.: 15/479,592

(22) Filed: Apr. 5, 2017

Related U.S. Application Data
(60) Provisional application No. 62/318,901, filed on Apr. 6, 2016.

(51) Int. Cl. C12M 1/12; C12M 1/06; C12M 1/00

(52) U.S. Cl. CPC C12M 23/06; C12M 29/14; C12M 27/02; C12M 27/20

(57) ABSTRACT

Disclosed herein are flow-through apparatuses for delivering microcarriers, the apparatuses comprising a body having a fixed volume and comprising an inlet, an outlet, and a cavity configured to contain at least one microcarrier; a first seal attached to the inlet and comprising at least one inlet port; a second seal attached to the outlet and comprising at least one outlet port; and at least one conduit attached to the at least one inlet port and/or the at least one outlet port. Systems comprising such flow-through apparatuses in fluid contact with a bioreactor are also disclosed herein. Further disclosed herein are methods for delivering microcarriers into a reactor, the methods comprising placing a delivery apparatus comprising at least one microcarrier in fluid contact with the reactor; and applying a force sufficient to induce flow of the at least one microcarrier into the reactor.

### CROSS-REFERENCE TO RELATED APPLICATIONS

This application claims the benefit of priority of U.S. Provisional Application Ser. No. 62/318,901 filed on Apr. 6, 2016 the content of which is relied upon and incorporated herein by reference in its entirety.

### FIELD OF THE DISCLOSURE

The present disclosure relates generally to apparatuses and systems for storing and delivering microcarriers, and more specifically to fixed volume apparatuses for delivering microcarriers into a bioreactor.

### BACKGROUND

Cell culture can be an important step in many human and animal treatments and therapies, for example, stem cell therapy and/or vaccine production, to name a few applications. Recent developments in cell culture have shown that it is possible to culture cells on microcarriers, e.g., in bioreactors. However, the scalability and efficiency of such processes are still relatively low and in need of improvement. In particular, the ability to aseptically package and transfer sterilized microcarrier beads for use in large-scale bioreactors has been problematic.

Current microcarrier delivery vessels include, for example, flexible media bags, which can be drained into the bioreactor using gravity and large amounts of liquid medium. Such media bags can, however, pose several disadvantages. First, gravity draining of the flexible media bag may leave residual microcarriers in the bag, which can result in wasted material and/or reduced process efficiency. Use of a pump or other mechanism to force microcarriers out of the bag has not presented a suitable alternative because the pumping action can crush the microcarriers. Moreover, the media bag often has a large volume (e.g., 5L or greater), but may contain low amounts of microcarriers (e.g., less than 300 g) due to the need to add liquid media to the bag for drainage of the carriers into the bioreactor. Thus, the media bag can be large and unwieldy for handling by the user. The lack of structural rigidity can also make user manipulation more difficult both before and after filling with liquid media. Further difficulty may arise during the manufacture and filling of such media bags, e.g., when pouring beads into a small opening in a large bag, which can cause static charge and clinging of the microcarrier beads to the outside of the bag.

Accordingly, it would be advantageous to provide methods and systems for improving the efficiency and/or sterility of microcarrier delivery into bioreactors. It would also be advantageous to provide methods for improving the ease of filling and emptying microcarriers from a delivery apparatus, e.g., for reducing material loss or product damage during filling and emptying of the apparatus.

### SUMMARY

The disclosure relates, in various embodiments, to flow-through apparatuses for delivering microcarriers, the apparatuses comprising a body having a fixed volume and comprising an inlet, an outlet, and a cavity configured to contain or containing at least one microcarrier; a first seal attached to the inlet and comprising at least one inlet port; a second seal attached to the outlet and comprising at least one outlet port; and at least one conduit attached to the at least one inlet port and/or the at least one outlet port. Systems comprising such flow-through apparatuses in fluid contact with a bioreactor are also disclosed herein.

Further disclosed herein are methods for delivering microcarriers into a reactor, the methods comprising placing a delivery apparatus comprising at least one microcarrier in fluid contact with the reactor; and applying a force sufficient to induce flow of the at least one microcarrier into the reactor; wherein the delivery apparatus comprises a body having a fixed volume and comprising an inlet, an outlet, and a cavity containing at least one microcarrier; a first seal attached to the inlet and comprising at least one inlet port; a second seal attached to the outlet and comprising at least one outlet port; and at least one conduit attached to the at least one inlet port and/or the at least one outlet port.

Flow-through apparatuses produced as set forth herein may deliver microcarriers to a bioreactor not only by gravity feed, but also using pumping or vacuum methods not readily applicable to currently marketed media bags. The application of force via a pump and/or vacuum can force media through the delivery apparatus such that the microcarriers are entrained in the medium and carried into the reactor. Thus, the delivery apparatuses disclosed herein can be relatively smaller because large amounts of liquid media need not be contained in the apparatus itself (e.g., as in the case of gravity draining media bags). The ratio of media to microcarrier can also be significantly reduced. Apparatuses disclosed herein can have increased structural rigidity as compared to a flexible media bag, thereby allowing for ease of manipulation by the user. The structural rigidity can also provide the user with a wider variety of methods for placing the apparatus in fluid contact with the bioreactor (e.g., as opposed to placing a bag on a hook above the bioreactor).

The apparatus can furthermore operate as a closed system by way of the first and second seals, such that the inlet and outlet ports can be customized as desired by the end-user, while still aseptically maintaining the microcarriers in the sterile body. Still further advantages can be provided when filling the apparatus as compared to pouring microcarriers into a small opening in a collapsed bag. It should be noted, however, that one or more of such characteristics may not be present according to various embodiments of the disclosure, yet such embodiments are intended to fall within the scope of the disclosure.

Additional features and advantages of the invention will be set forth in the detailed description which follows, and in part will be readily apparent to those skilled in the art from that description or recognized by practicing the invention as described herein, including the detailed description which follows, the claims, and the appended drawings.

It is to be understood that both the foregoing general description and the following detailed description present various embodiments of the disclosure, and are intended to provide an overview or framework for understanding the nature and character of the claims. The accompanying drawings are included to provide a further understanding of the disclosure, and are incorporated into and constitute a part of this specification. The drawings illustrate various embodiments of the disclosure and together with the description serve to explain the principles and operations of the invention.

### BRIEF DESCRIPTION OF THE DRAWINGS

The following detailed description can be best understood when read in conjunction with the following drawings, in which:

FIG. 1 is a schematic illustrating a delivery apparatus according to various embodiments disclosed herein;

FIG. 2 is a cross-sectional view of a delivery apparatus according to additional embodiments of the disclosure;

FIG. 3A is a cross-sectional view of a delivery apparatus according to further embodiments of the disclosure;

FIG. 3B is a cross-sectional view of the delivery apparatus of FIG. 3A taken along line A-A;

FIG. 4 is a schematic illustrating a delivery apparatus according to various embodiments disclosed herein;

FIG. 5 is a cross-sectional view of a delivery apparatus according to certain embodiments of the disclosure;

FIGS. 6A-B are cross-sectional views of a delivery apparatus according to additional disclosed embodiments;

FIG. 7 is a cross-sectional view of a delivery apparatus according to further embodiments of the disclosure;

FIG. 8 is a schematic of a bioreactor system according to the prior art; and

FIG. 9 is a schematic of a bioreactor system according to certain embodiments of the disclosure.

### DETAILED DESCRIPTION

Apparatuses

Disclosed herein are flow-through apparatuses for delivering microcarriers, the apparatuses comprising a body having a fixed volume and comprising an inlet, an outlet, and a cavity configured to contain or containing at least one microcarrier; a first seal attached to the inlet and comprising at least one inlet port; a second seal attached to the outlet and comprising at least one outlet port; and at least one conduit attached to the at least one inlet port and/or the at least one outlet port. Systems comprising such flow-through apparatuses in fluid contact with a bioreactor are also disclosed herein.

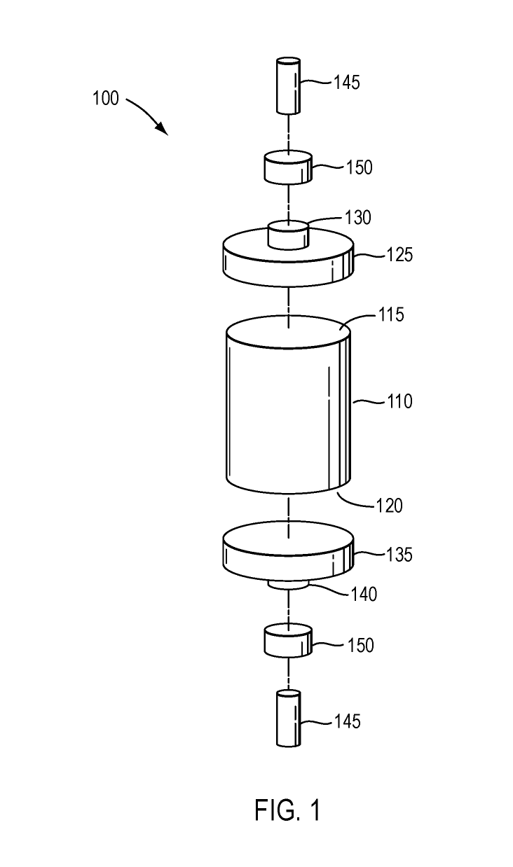

Embodiments of the disclosure will be discussed with reference to FIG. 1, which illustrates a schematic of a delivery apparatus according to non-limiting embodiments of the disclosure. The following general description is intended to provide an overview of the claimed apparatus and various aspects will be more specifically discussed throughout the disclosure with reference to the non-limiting embodiments, these embodiments being interchangeable with one another within the context of the disclosure.

As demonstrated in FIG. 1, a delivery apparatus 100 can comprise a body 110 having a fixed volume and comprising an inlet 115, an outlet 120, and a cavity containing at least one microcarrier (not illustrated). A first seal 125 comprising at least one inlet port 130 can be attached to the inlet 115 and a second seal 135 comprising at least one outlet port 140 can be attached to the outlet 120. At least one conduit 145 can be attached to one or more of the inlet and/or outlet port(s). The apparatus can further comprise one or more locks 150, such as a barb lock on the inlet and/or outlet port(s) 130, 140. While FIG. 1 illustrates an apparatus comprising one inlet port 130 and one outlet port 140, with one conduit 145 to each of the inlet and outlet ports, it is to be understood that the first and second seals 125, 135 can comprise any number of ports, and any number of conduits can be attached to the ports as desired for operation. Furthermore, while FIG. 1 depicts the apparatus in a vertical alignment, with the inlet 115 positioned above the outlet 120, it is to be understood that the apparatus may have any orientation, and the inlet and outlet ports as labeled can be interchanged as appropriate for the desired process flow.

The apparatuses 100 of the present disclosure can comprise a body 110 having a fixed volume. As used herein, the term “fixed volume” and variations thereof is intended to denote that the body is substantially non-compressible, e.g., substantially rigid. Whereas the volume of a compressible bag can change with the addition of liquid media, the apparatuses disclosed herein maintain substantially the same internal volume regardless of the addition of media or other substances to the cavity.

The body can have any shape desired, such as a cylinder, cube, sphere, or any other suitable, regular or irregular, three-dimensional shape. In non-limiting embodiments, the body 110 can have a cylindrical shape. The body 110 can comprise any material suitable for storing and delivering microcarriers, e.g., a substantially inert and/or non-reactive material with respect to the microcarriers, such as non-leachable and/or non-extractable materials. Suitable materials can include, for example, plastics, metals, polymers, glass, and the like. According to various embodiments, the body can comprise a plastic, such as polystyrene, polyethylene, or polypropylene, which can provide structural rigidity to the apparatus. In certain embodiments, the body 110 can be fully or partially constructed from an optically transparent material such that the contents placed therein are visible to the user. For example, the entire body may be transparent, or a transparent window or strip can be provided on an otherwise opaque body. Furthermore, in additional embodiments, markers or tick marks can be provided on the exterior or interior of the body 110 such that the user can monitor and/or measure the amount of media introduced into the apparatus and/or exiting the apparatus.

The body 110 can comprise a cavity, or hollow portion, which can contain at least one microcarrier. The cavity can likewise have any desired shape, such as a cylindrical or spherical cavity, or the like, as appropriate for the desired application. According to various embodiments, the body 110 can comprise a cylinder with a cylindrical cavity. The cavity can contain the at least one microcarrier, which can be provided in any form suitable for delivery to a bioreactor. For instance, in non-limiting embodiments, the at least one microcarrier can be suspended in a liquid or gaseous medium. The suspension can comprise, for example, from about 10% to about 95% by weight of microcarriers, such as from about 20% to about 90%, from about 30% to about 80%, from about 40% to about 70%, or from about 50% to about 60% by weight of microcarriers, including all ranges and subranges therebetween. The liquid medium can be chosen, in some embodiments, from water, cell media, and combinations thereof. Gaseous media can include, for instance, air and inert or noble gases such as nitrogen, argon, helium, and the like. In additional embodiments, the cavity can comprise solid microcarrier particles (or beads) in the absence of a liquid medium, e.g., “dry” or substantially dry microcarriers. For example, dry microcarriers can comprise less than about 5% by weight of liquid relative to the total weight of microcarriers, such as less than about 4%, 3%, 2%, 1%, 0.5%, 0.1%, or 0.01% by weight of liquid, or 0% liquid, including all ranges and subranges therebetween.

As used herein, the term “microcarrier” and variations thereof is intended to refer to particles configured for cell growth on their surface. Microcarriers can be created out of a variety of materials, such as plastics, polymers, glass, gelatin, and calcium alginate, in order to increase the surface area available for cell growth (e.g., as compared to one-dimensional or two-dimensional growth in a tube or flask). Microcarriers can be in the form of beads, e.g., substantially spherical beads, but can also take any other suitable shape, such as regular or irregular shapes including, but not limited to, ovoid shapes. Non-limiting examples of commercially available microcarriers include, for example, Cytodex™ (GE Healthcare) or SoloHill® (Pall).

In various embodiments, the microcarriers, either with or without added medium, can substantially fill the cavity. For instance, the cavity can comprise less than about 5% headspace in certain embodiments. The term “headspace” as used herein is intended to refer to empty, unfilled volume in the apparatus cavity. According to various embodiments, the cavity can comprise less than about 4%, 3%, 2%, or 1% headspace, including all ranges and subranges therebetween. In a non-limiting embodiment, the cavity can be fully filled with microcarriers, either in suspension or as dry, solid particulates, e.g., 0% headspace. According to still further embodiments, the cavity can comprise greater than about 5% headspace, such as about 10%, 15%, 20%, 25%, 30%, 35%, 40%, 45%, 50% headspace, or greater, including all ranges and subranges therebetween. For example, the cavity can contain dry microcarriers (e.g., no liquid medium) and less than about 50% headspace, such as less than about 40%, 30%, 20%, 10%, 5%, 3%, 2%, or 1% headspace, including all ranges and subranges therebetween. The apparatus cavity can also, in certain embodiments, be completely (100%) full of dry microcarriers.

The dimensions of the apparatus body 110 can vary as appropriate for process scalability. For example, the body and cavity can be sized to meet user packaging requirements, such as 100 g, 500 g, 1 kg, etc. of microcarriers. The desired amount of headspace can also be varied as desired and can impact the final shape and/or size of the cavity. In certain embodiments, the apparatus body can comprise an open cylinder having a diameter ranging from about 2 cm to about 15 cm, such as from about 3 cm to about 12 cm, from about 4 cm to about 10 cm, or from about 5 cm to about 8 cm, and a length ranging from about 10 cm to about 50 cm, such as from about 20 cm to about 40 cm, or from about 25 cm to about 30 cm, including all ranges and subranges therebetween.

The inlet 115 of the body 110 can be fitted with a first seal 125 comprising one or more inlet ports 130. Similarly, the outlet 120 of the body 110 can be fitted with a second seal 135 comprising one or more outlet ports 140. The first and second seals can be identical or different and can, in some embodiments, comprise end caps that can be reversibly attached to the inlet and outlet, respectively. The seals can comprise, for example, circular or conical caps comprising one or more ports. As shown in FIG. 1, the first and second seals 125, 135 are identical, comprising circular caps each having one port 130, 140. However, the seals can be different from one another and can have any suitable shape other than that depicted. For example, the seals can have a conical shape converging at the one or more ports, such as a funnel-shape, which may, in various embodiments, provide more efficient microcarrier transfer. The seals and their respective ports can comprise any material suitable for the storage and transfer of microcarriers, e.g., a material similar to or different from that of the body 110, such as plastics, metals, glass, polymers, and the like. According to various embodiments, the first and second seals 125, 135 can comprise a plastic, such as polystyrene, polypropylene, or polytetrafluoroethylene.

In additional embodiments, the first and second seals 125, 135 can respectively comprise one or more inlet and outlet ports 130, 140. For example, each of the first and second seals can comprise one, two, three, four, five, or more ports, as desired. Moreover, the number of inlet ports 130 may not necessarily be equal to the number of outlet ports 140. One or more of the ports 130, 140 may comprise a conduit 145 for delivery of a medium into the apparatus (e.g., a conduit attached to an inlet port), or for delivery of microcarriers and/or media out of the apparatus (e.g., a conduit attached to an outlet port). The number of conduits 145 attached to the inlet and outlet ports can vary and need not match the number of ports available (which can, for example, be sealed). The seals can, in certain embodiments, further comprise a lock 150 for attaching one or more conduits 145 to the inlet and outlet ports 130, 140. Locks can include barb lock retainers (e.g., BarbLock® from Saint-Gobain) or any other suitable connective fitting for securing a conduit, such as a tube, to a port. Each of the conduits and locks can comprise suitable materials as described herein, e.g., plastics, polymers, etc.

The delivery apparatuses disclosed herein can, in certain embodiments, be configured for re-sealing after use or partial use. For instance, a user can deliver a desired amount of microcarriers and/or media to the bioreactor after which the user can seal the apparatus and remove it from the system for future use. In some embodiments, a valve, clamp, or other feature can be included on the inlet or outlet ports to stop or restrict flow of media and/or microreactors. Alternatively, one or both of the first or second seals can have a valve, clamp, or similar feature. Other additional features can be incorporated in the delivery apparatus, such as filters positioned at the inlet or outlet of the delivery apparatus. Furthermore, such features, caps, and tubes can be interchangeable as desired by the user while still maintaining the microcarriers in an aseptic environment.

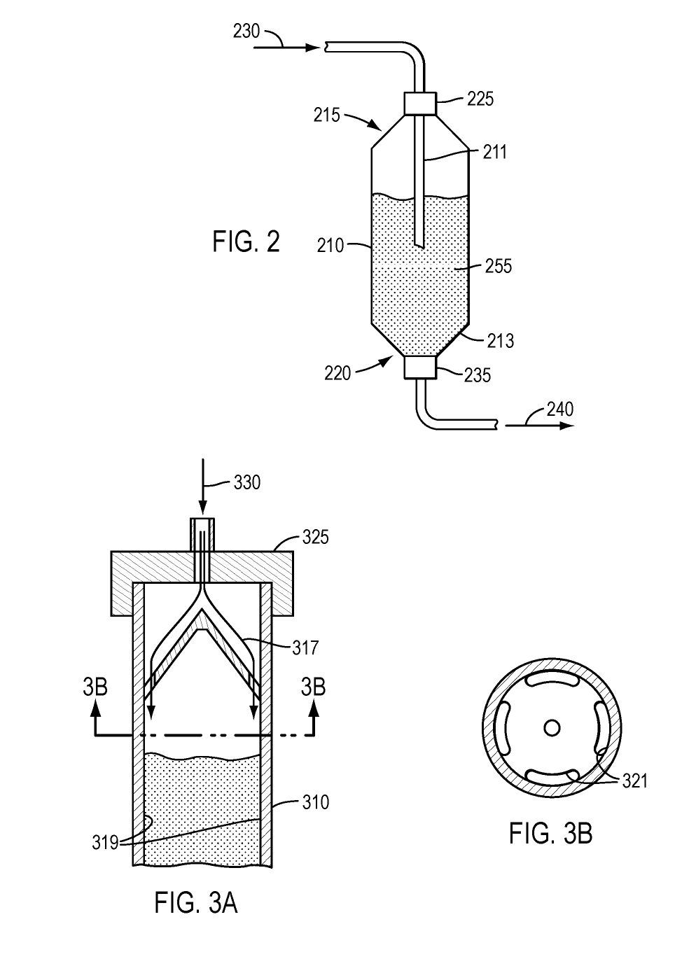

In additional embodiments, the apparatus can be equipped with one or more features configured to enhance flow and/or mixing of the microcarriers within the apparatus. For example, the delivery apparatus can be configured to promote laminar and/or turbulent flow within the apparatus body. Such configurations can, in some embodiments, reduce the likelihood of clogging, e.g., by dense and/or compact pockets of microcarriers poorly dispersed by the fluid. These and other features will be discussed with reference to FIGS. 2-7, it being understood that any features can be included in any apparatus disclosed herein, alone or in combination, and without limitation.

FIG. 2 depicts a cross-sectional view of an apparatus according to various non-limiting embodiments of the disclosure comprising an inlet 215, an outlet 220, inlet and outlet ports 230, 240, and first and second seals 225, 235. In the depicted embodiment, the inlet 215 can be equipped with a tube 211, such as a dip tube or other similar conduit, which can be used to offset the introduction of media into the body 210 of the delivery apparatus. For example, the tube 211 can extend partially into the body to provide flow in a more central position within the body 210. In some embodiments, the tube 211 can extend into the body at a distance approximately equal to about 10% or more of the length of the body, such as about 20%, 30%, 40%, 50%, 60%, 70%, 80%, 90%, or more, of the length of the body 210. According to various embodiments, as illustrated in FIG. 2, the dip tube can extend about halfway (50%) along the length of the body 201. Introduction of the media at a more central location, e.g., at a given distance away from the inlet 215 of the body, can promote mixing of the media with the microcarriers 255.

Mixing can also be promoted by the contour or shape of the body 210, as shown in FIG. 2. For example, the body can be shaped to remove or reduce any sharp angles, e.g., by beveling one or both of the inlet 215 or outlet 220 to produce tapered ends 213. Internal geometries with sharp angles can produce dead space, e.g., regions with reduced fluid flow and/or mixing, in which the microcarriers 255 might become trapped. According to various embodiments, the internal surface of the body can have at least one rounded or beveled contour, such as a bullet-shape, or all corners of the body interior can be rounded or beveled, such as an ovoid shape. While FIG. 2 illustrates a delivery apparatus also having a tapered external shape, it is possible that the exterior of the body can have one shape, e.g., a cylinder or the like, and the interior surface of the body can have a different shape, e.g., having rounded or tapered interior surfaces. Of course any interior (or exterior) geometries can be used to achieve the desired mixing effect. Rounding or contouring of the delivery apparatus can, in some embodiments, reduce or eliminate dead spaces and promote flow of the microcarriers 255 into the bioreactor.

Additional configurations for promoting mixing and/or laminar flow within the delivery apparatus are depicted in FIGS. 3A-B. As shown in FIG. 3A, one or more flow distribution components 317, such as cones, baffles, and the like, can be positioned proximate the inlet port 330 or cap 325. As media flows into the body 310, illustrated by the downward pointing arrows, it can contact the distribution component 317 and disperse along the side walls 317 of the body 310. FIG. 3B illustrates a cross-sectional view of the apparatus of FIG. 3A, as viewed along line A-A. As depicted, the distribution component 317 can comprise one or more holes 319 through which media can flow to enhance distribution of the media throughout the body 310. Of course, other distribution components can be employed to achieve different fluid flow patterns as desired. Moreover, while FIG. 3A depicts a vertically-oriented delivery apparatus and corresponding distribution component 317, horizontally-oriented delivery apparatuses and distribution components are also contemplated by and considered part of the instant disclosure.

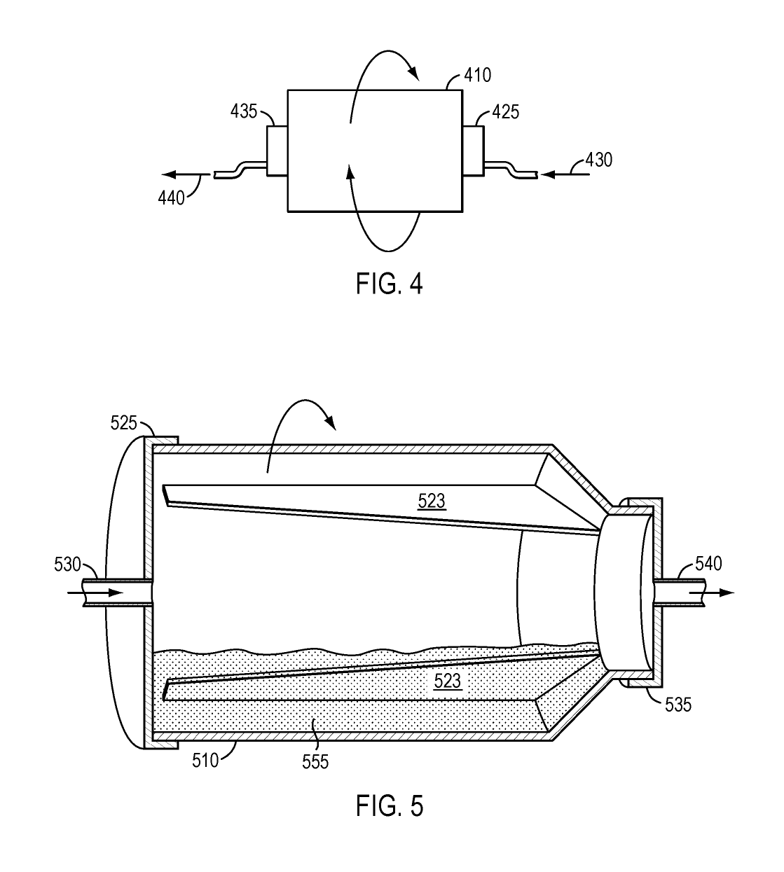

In further embodiments, the delivery apparatus can be equipped with various features for promoting turbulence in the delivery apparatus. For instance, as depicted in FIG. 4, the apparatus can be configured to rotate about an axis, e.g., around a horizontal axis in the direction indicated by the arrows. Of course, the apparatus can also be configured to rotate around a vertical axis if desired. In some embodiments, the inlet and outlet ports 430, 440 and first and second seals 425, 435 can be stationary, e.g., not rotatable about the axis, while the body 410 can be rotated around the axis. Rotation can be achieved manually, e.g., a user can spin or rotate the body by hand at any desired time or repeated time interval. Alternatively, the body 410 can be mechanically rotated at any desired time or repeated time interval.

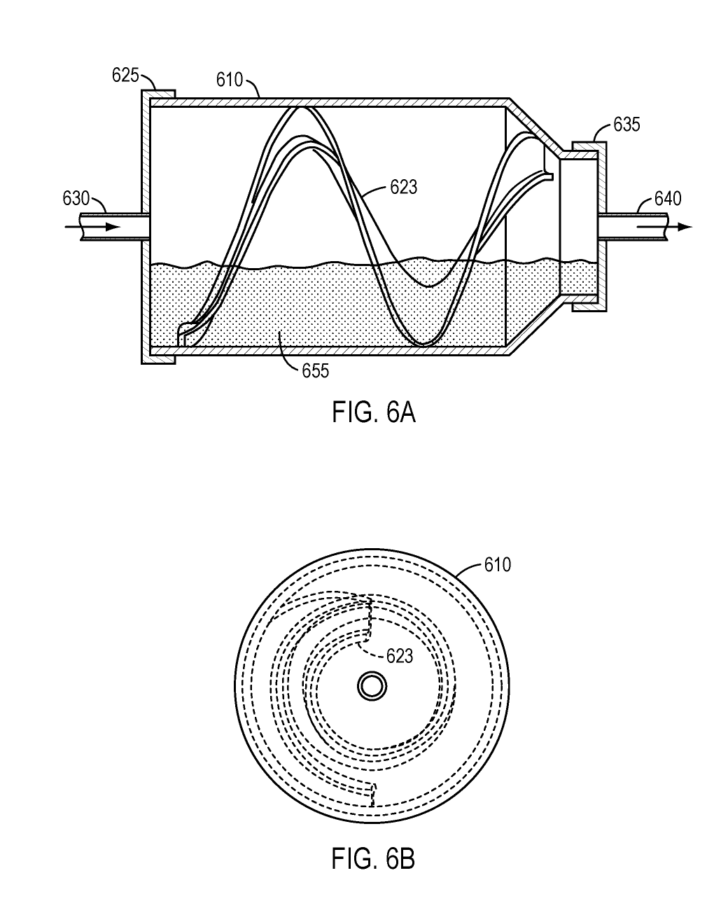

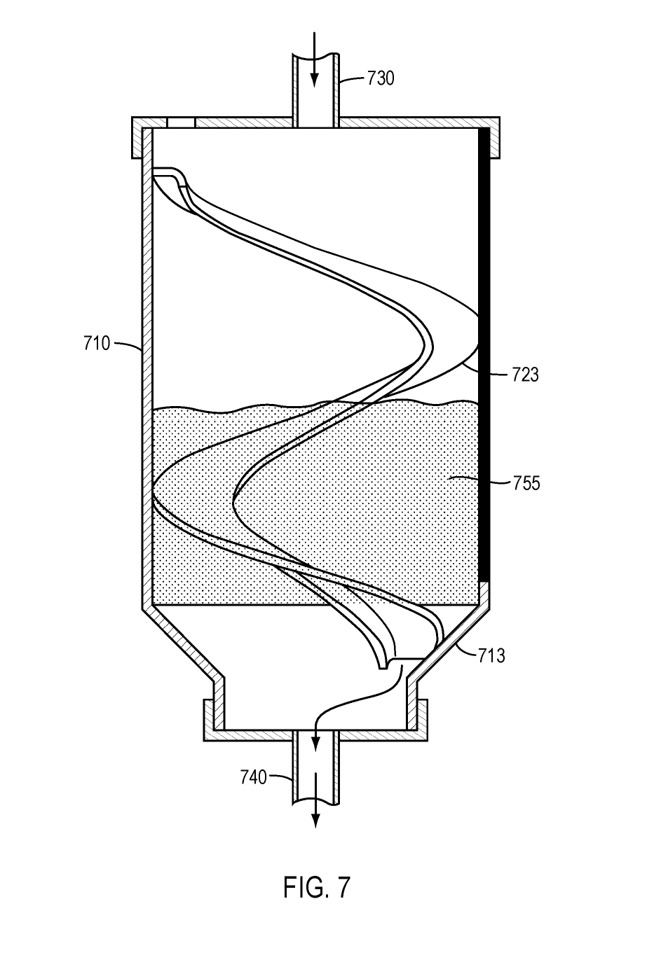

Various features can be included on the interior surfaces of the delivery apparatus to promote turbulent flow, alone or in combination with the rotatable feature depicted in FIG. 4. For example, as depicted in FIG. 5, one or more blades 523 can be included inside the body 510. The blades 523 can be stationary, e.g., fixedly attached to the side walls of the body 510, or rotatable, e.g., rotatable attached to any interior feature of the body 510, such as the seals 525 or 535. As the body 510 and/or the blade(s) 523 rotate, the microcarriers 555 inside the delivery apparatus can be mixed and dispersed by the blade(s) 523. Inlet and outlet ports 530, 540 can, in some embodiments, remain stationary during rotation and mixing. The blades 523 can be substantially planar or two-dimensional as depicted in FIG. 5 or they can be non-planar or three-dimensional as depicted in FIG. 6A. For instance, the body 610 can comprise a helical blade 623 for mixing the microcarriers 655, e.g., during rotation of the body 610 and/or blade 623. As in FIG. 5, the first and second seals 625, 635 and/or inlet and outlet ports 630, 640 can remain stationary during rotation. The blade 623 can have any configuration or proportion as desired to promote mixing and/or turbulent flow within the delivery apparatus. In some embodiments, as illustrated in FIG. 6B, the blade 623 can be tapered to promote mixing of the microcarriers with the medium and movement of the mixture out of the delivery apparatus.

While FIG. 6A depicts a horizontally-oriented delivery apparatus, it is also possible to employ a helical blade in a vertically-oriented delivery apparatus, as illustrated in FIG. 7. For instance, fluid introduced into one or more inlet ports 730 can flow into the body 710 and come into contact with the helical blade 723 which can serve as a stairway or path down which the media can flow. The helical blade 723 can also function to direct the microcarriers 755 out of the delivery apparatus via outlet port 740. In some embodiments, the helical blade 723 can be stationary (e.g., serving as a baffle similar to the embodiment depicted in FIG. 3A) or can be rotatable (e.g., serving as a turbulent mixer similar to the embodiment depicted in FIG. 6A). The body 710 can also, in some embodiments, be rotatable, e.g., in conjunction with a fixed or rotatable blade 723.

As illustrated in FIG. 7, and as discussed with reference to FIG. 2, one or more regions of the apparatus can be tapered to promote fluid flow within the delivery apparatus. For instance, tapered edges 713 can be provided proximate the outlet port 740 to reduce or eliminate dead space in which the microcarriers 755 might be caught. In some embodiments, tapered edges can also be provided proximate the inlet port 730. According to other embodiments, as illustrated in FIG. 7, tapered edges may not be provided proximate the inlet for a vertically-oriented delivery apparatus. Other features may also be included in the case of vertically-oriented delivery apparatuses, such as hooks or handles proximate the inlet for hanging or otherwise suspending the delivery apparatus.

The apparatuses disclosed herein can be used for the sterile or aseptic transfer of microcarriers into a bioreactor. As such, after filling the cavity with at least one microcarrier, the apparatus can be sealed and sterilized prior to placing the apparatus in contact with the bioreactor. According to various embodiments, the apparatus components can be sterilized separately or together prior to assembling the device and filling it with the microcarriers. In additional embodiments, the apparatus can be sterilized after it is filled with microcarriers. Sterilization of the apparatus or its individual components can be carried out by gamma irradiation, ethylene oxide, E-beam, or steam sterilization, and combinations thereof. As such, in contrast to the free addition of microcarriers to a bioreactor (e.g., by pouring microcarriers directly into the reactor), microcarriers packaged in the instantly disclosed apparatuses can be maintained in an aseptic condition just prior to and during their addition to the reactor.

Systems and Methods

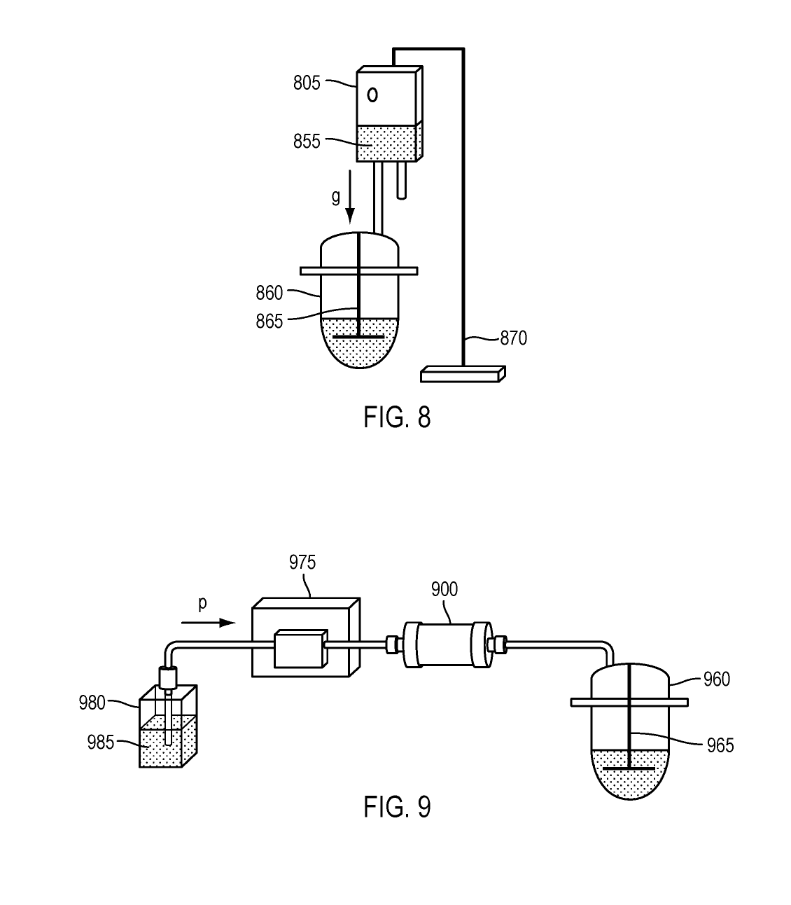

The delivery apparatuses disclosed herein can be employed in a system comprising a bioreactor, e.g., for cell culture. FIG. 8 depicts a prior art system comprising delivery of microcarriers into a reactor. As previously discussed, prior art delivery apparatuses can include a flexible bag 805 comprising a suspension 855 of microcarriers in liquid media. The bag 805 is suspended over the bioreactor 860 by way of stand 870 (e.g., hooked to the stand), such that gravity flow g draws the microcarriers and liquid media into the bioreactor 860, which can optionally be fitted with an agitator 865. The flexible bag 805 as received by the user may comprise a small amount of microcarriers, to which the user may add liquid media to make the suspension 855. The bag volume must therefore have capacity (e.g., headspace) to receive the volume of liquid necessary to transfer the microcarriers into the bioreactor 860 by way of gravity flow g.

In contrast, referring to FIG. 9, which depicts an exemplary system according to the instant disclosure, the delivery apparatus 900 can be placed in fluid contact with a bioreactor 960 (optionally comprising agitator 965) such that microcarriers can flow into the bioreactor for further processing via gravity flow, pressurized flow, and/or vacuum flow. As depicted in FIG. 9, the apparatus 900 can, in some embodiments, be fitted in-line upstream of the bioreactor 960. In the case of a system comprising a pump 975 (as depicted), the apparatus 900 can be fitted in-line downstream of the pump 975 and upstream of the reactor 960. A vessel 980 can contain liquid media 985, which can be pumped through the apparatus 900. The apparatus 900 can therefore comprise dry microcarriers or microcarriers in suspension (not labeled), to which the media 985 is added. The microcarriers entrained in the media can thus flow into the bioreactor by way of pressurized flow p. Similarly, although not illustrated, a vacuum can be placed in-line in the system to draw microcarriers out of the apparatus and into the reactor, e.g., by drawing a medium through the apparatus.

Methods disclosed herein can comprise placing a delivery apparatus comprising at least one microcarrier (as described herein) in fluid contact with a reactor; and applying a force sufficient to induce flow of the at least one microcarrier into the reactor. The delivery apparatus can be configured for gravity flow, vacuum flow, and/or pressurized flow of the at least one microcarrier out of the cavity through the at least one outlet port. Force, such as gravity, can be applied by placing the delivery apparatus in an elevated position with respect to the reactor. Force, such as positive pressure, can be applied by at least one pump configured for pressurized flow of a medium and/or the microcarriers through the delivery apparatus. Force, such as negative pressure, can be applied by at least one vacuum configured for vacuum flow of a medium and/or the microcarriers through the delivery apparatus. For example, an apparatus comprising microcarriers, e.g., dry microcarriers, can be placed in fluid contact with a reactor and a liquid can be introduced into the cavity. The liquid can, for instance, be pumped or sucked through the flow-through apparatus, thereby entraining the microcarriers and carrying them into the reactor. In additional embodiments, liquid can be added to the cavity and the cavity can then be gravity drained into the reactor.

According to various embodiments, the methods disclosed herein can further comprise a step of introducing the at least one microcarrier into the delivery apparatus. The methods can comprise, for example, attaching one of the first or second seal to the inlet or outlet of the body, respectively, introducing the at least one microcarrier into the cavity, attaching the other one of the first or second seal to the body, and optionally sterilizing the delivery apparatus. In additional embodiments, the delivery apparatus can be placed into fluid contact with the reactor by connecting the at least one conduit to the reactor.

As used herein, the term “fluid contact” and variations thereof is intended to denote that a substance, e.g., microcarriers and/or medium can freely flow from the delivery apparatus to the reactor without obstruction. Fluid contact may be blocked and/or reestablished by closing and/or opening one or more components of system, e.g., by closing a valve or blocking or clamping a conduit, or vice versa. Similarly, the term “flow-through” and variations thereof is intended to denote that a substance, e.g., microcarriers and/or medium can freely flow through an apparatus via the inlet and outlet ports without obstruction. A flow-through apparatus can be placed in-line in a system in a closed orientation and can be subsequently opened when fluid flow through the apparatus is desired.

While the system depicted in FIG. 9 illustrates introduction of media 985 into the bioreactor via a single conduit, e.g., after flowing through the delivery apparatus 960, it is to be understood that media can be introduced into the bioreactor via more than one conduit. For instance, additional media can be introduced into the bioreactor 960 via at least one additional conduit, e.g., without first flowing the media through the delivery apparatus 900. In alternative embodiments, media 985 can be flushed through the delivery apparatus 900 to entrain and carry the microbeads into the bioreactor 960 and, subsequently, additional media can be pumped into the bioreactor through the same (or different) flow path if desired.

It will be appreciated that the various disclosed embodiments may involve particular features, elements or steps that are described in connection with that particular embodiment. It will also be appreciated that a particular feature, element or step, although described in relation to one particular embodiment, may be interchanged or combined with alternate embodiments in various non-illustrated combinations or permutations.

It is also to be understood that, as used herein the terms “the,” “a,” or “an,” mean “at least one,” and should not be limited to “only one” unless explicitly indicated to the contrary. Thus, for example, reference to “a solvent” includes examples having two or more such “solvents” unless the context clearly indicates otherwise.

Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All numerical values expressed herein are to be interpreted as including “about,” whether or not so stated, unless expressly indicated otherwise. It is further understood, however, that each numerical value recited is precisely contemplated as well, regardless of whether it is expressed as “about” that value. Thus, “a dimension less than 10 mm” and “a dimension less than about 10 mm” both include embodiments of “a dimension less than about 10 mm” as well as “a dimension less than 10 mm.”

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method comprising A+B+C include embodiments where a method consists of A+B+C, and embodiments where a method consists essentially of A+B+C.

It will be apparent to those skilled in the art that various modifications and variations can be made to the present disclosure without departing from the spirit and scope of the disclosure. Since modifications combinations, sub-combinations and variations of the disclosed embodiments incorporating the spirit and substance of the disclosure may occur to persons skilled in the art, the disclosure should be construed to include everything within the scope of the appended claims and their equivalents.

What is claimed is:

1. A flow-through apparatus for delivering microcarriers comprising:
(a) a body having a fixed volume and comprising an inlet, an outlet, and a cavity configured to contain at least one microcarrier;
(b) a first seal attached to the inlet and comprising at least one inlet port;
(c) a second seal attached to the outlet and comprising at least one outlet port; and
(d) at least one conduit attached to the at least one inlet port and/or the at least one outlet port.

2. The apparatus of claim 1, wherein the apparatus is configured for gravity flow, vacuum flow, and/or pressurized flow of the at least one microcarrier out of the cavity through the at least one outlet port.

3. The apparatus of claim 1, wherein the body is a substantially rigid cylinder.

4. The apparatus of claim 1, wherein at least one of the first and second seals comprises a snap cap and lock and wherein the at least one conduit comprises a tube.

5. The apparatus of claim 1, wherein the cavity comprises at least one mixing blade.

6. The apparatus of claim 5, wherein the at least one mixing blade is planar or helical.

7. The apparatus of claim 5, wherein the at least one mixing blade is fixedly attached to an interior surface of the body.

8. The apparatus of claim 5, wherein the at least one mixing blade is rotatably attached to the apparatus.

9. The apparatus of claim 1, wherein the body is rotatably attached to the first and second seals.

10. The apparatus of claim 1, wherein and interior surface of the body comprises at least one rounded or bevelled contour.

11. The apparatus of claim 1, wherein the inlet of the body further comprises a dip tube extending into the cavity for a distance of at least about 10% of a length of the body.

12. The apparatus of claim 1, further comprising at least one baffle in the cavity proximate the inlet.

13. A system comprising the apparatus of claim 1 in fluid contact with a bioreactor.

14. The system of claim 13, wherein the apparatus is connected to the bioreactor by the at least one conduit.

15. The system of claim 13, wherein the apparatus is in an elevated position relative to the bioreactor and wherein the at least one microcarrier flows into the bioreactor by gravity flow.

16. The system of claim 13, further comprising a pump configured for pressurized flow of a medium through the apparatus, wherein the at least one microcarrier is entrained in the medium and introduced into the bioreactor by the pressurized flow.

17. The system of claim 16, wherein the medium is a liquid or gaseous medium.

18. The system of claim 13, wherein the bioreactor further comprises at least one additional conduit.

19. A method for delivering microcarriers into a reactor, comprising:
placing a delivery apparatus comprising at least one microcarrier in fluid contact with the reactor;
applying a force sufficient to induce flow of the at least one microcarrier into the reactor, wherein the delivery apparatus comprises:
(a) a body having a fixed volume and comprising an inlet, an outlet, and a cavity configured to contain the at least one microcarrier;
(b) a first seal attached to the inlet and comprising at least one inlet port;
(c) a second seal attached to the outlet and comprising at least one outlet port; and
(d) at least one conduit attached to the at least one inlet port and/or the at least one outlet port.

20. The method of claim 19, wherein the force is chosen from gravity, pressure, vacuum, and combinations thereof.

21. The method of claim 20, wherein gravity is applied by placing the delivery apparatus in an elevated position with respect to the reactor.

22. The method of claim 20, wherein pressure is applied by at least one pump configured for pressurized flow of a medium through the delivery apparatus, wherein the at least one microcarrier is entrained in the medium.

23. The method of claim 20, wherein the medium is a liquid or gaseous medium.

24. The method of claim 20, further comprising introducing additional medium into the bioreactor via a separate conduit.

25. The method of claim 19, further comprising introducing the at least one microcarrier into the delivery apparatus by:
(a) attaching one of the first or second seal to the body,
(b) introducing the at least one microcarrier into the cavity,
(c) attaching the other one of the first or second seal to the body, and
(d) optionally sterilizing the delivery apparatus.
